SEQUENCE LISTING (13 sequences)

SEQ ID NO: 1 - protein, 122 aa, synthetic construct
EVQLVESGGGLIQPGRSLRLSCAASGITFDDAVMHWVRQAPGKGLEWVAGISSNSGYIGYADSVKGRFTISRDNAKNSLYLQMNRLRAEDTAVYYCVKGLYSNPRGGAFDIWGQGTMVTVSS

SEQ ID NO: 2 - protein, 8 aa, synthetic construct
GITFDDAV

SEQ ID NO: 3 - protein, 8 aa, synthetic construct
ISSNSGYI

SEQ ID NO: 4 - protein, 15 aa, synthetic construct
VKGLYSNPRGGAFDI

SEQ ID NO: 5 - protein, 110 aa, synthetic construct
SYVLTQPPSVSVAPGQTATITCGGNNIGTKSVHWYQQKPGQAPVLVVYADSDRPSGIPERVSGSNSGNTATLTISRVEAGDEADYYCQVWDSRSDHLWVFGGGTKLTVLG

SEQ ID NO: 6 - protein, 6 aa, synthetic construct
NIGTKS

SEQ ID NO: 7 - skipped

SEQ ID NO: 8 - protein, 12 aa, synthetic construct
QVWDSRSDHLWV

SEQ ID NO: 9 - protein, 253 aa, synthetic construct
EVQLVESGGGLIQPGRSLRLSCAASGITFDDAVMHWVRQAPGKGLEWVAGISSNSGYIGYADSVKGRFTISRDNAKNSLYLQMNRLRAEDTAVYYCVKGLYSNPRGGAFDIWGQGTMVTVSSASTGGGGSGGGGSGGGGSVHSSYVLTQPPSVSVAPGQTATITCGGNNIGTKSVHWYQQKPGQAPVLVVYADSDRPSGIPERVSGSNSGNTATLTISRVEAGDEADYYCQVWDSRSDHLWVFGGGTKLTVLG

SEQ ID NO: 10 - protein, 305 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPC

SEQ ID NO: 11 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVTSYLNAS

SEQ ID NO: 12 - protein, 510 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVTSYLNAS

SEQ ID NO: 13 - protein, 788 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVTSYLNASGGGGSGGGGSGGGGSGGGGSGGGGSEVQLVESGGGLIQPGRSLRLSCAASGITFDDAVMHWVRQAPGKGLEWVAGISSNSGYIGYADSVKGRFTISRDNAKNSLYLQMNRLRAEDTAVYYCVKGLYSNPRGGAFDIWGQGTMVTVSSASTGGGGSGGGGSGGGGSVHSSYVLTQPPSVSVAPGQTATITCGGNNIGTKSVHWYQQKPGQAPVLVVYADSDRPSGIPERVSGSNSGNTATLTISRVEAGDEADYYCQVWDSRSDHLWVFGGGTKLTVLG

(19) United States
(12) Patent Application Publication
(10) Pub. No.: US 2026/0234210 A1
(43) Pub. Date: Aug. 13, 2026

(54) METHODS OF MAKING RECOMBINANT IL-12 ALBUMIN BINDING DOMAIN FUSION PROTEINS

(71) Applicant: Guidant BioTherapeutics, Inc., Princeton, NJ (US)

(72) Inventors: Susan DEXTER, Rye, NH (US); Anagha NAVALE, Pune (IN); Deepali MAGADUM, Pune (IN); Abijar BHORI, Pune (IN); Veerendra Kumar REDDY, Pune (IN); Rutuja SHETH, Pune (IN); Shilpa GADGIL, Pune (IN); Himanshu GADGIL, Pune (IN)

(73) Assignee: Guidant BioTherapeutics, Inc., Princeton, NJ (US)

(21) Appl. No.: 19/164,812

(22) PCT Filed: Mar. 13, 2024
(86) PCT No.: PCT/US2024/019798

§ 371 Date: Sep. 12, 2025

Related U.S. Application Data
(60) Provisional application No. 63/490,202, filed on Mar. 14, 2023.

(51) Int. Cl. C07K 14/54; C07K 1/22; C07K 16/18; C12M 1/00; C12N 5/071

(52) U.S. Cl. CPC C07K 14/5434; C07K 1/22; C07K 16/18; C12M 29/10; C12M 47/12; C12N 5/0602; C07K 2317/56; C07K 2317/622; C07K 2319/01

(57) ABSTRACT

Provided herein are methods for making recombinant IL-12 albumin binding domain (ABD) fusion proteins. The subject methods advantageously include a continuous downstream purification step that leads to high yield production of purified cytokine-based proteins.

### CROSS-REFERENCE TO RELATED APPLICATIONS

The present application is a National Stage Application of International Patent Application No. PCT/US2024/019798, filed on Mar. 13, 2024, which claims priority to U.S. Provisional Patent Application No. 63/490,202, filed on Mar. 14, 2023, each of which are hereby incorporated by reference in their entirety.

### SEQUENCE LISTING

This application contains a sequence listing, which has been submitted in XML format via Patent Center. The contents of the XML copy named “116076-5016-US Updated,” which was created on Sep. 10, 2025, and is 13,927 bytes in size, are incorporated herein by reference in their entirety.

### BACKGROUND

IL-12 is known as a T cell-stimulating factor that can stimulate the growth and function of T cells. In particular, IL-12 can stimulate the production of interferon gamma (IFN-7), and tumor necrosis factor-alpha (TNF-α) from T cells and natural killer (NK) cells and reduce IL-4 mediated suppression of IFN-7. IL-12 can further mediate enhancement of the cytotoxic activity of NK cells and CD8+ cytotoxic T lymphocytes. Moreover, IL-12 can also have anti-angiogenic activity by increasing production of interferon gamma, which in turn increases the production of the chemokine inducible protein-10 (IP-10 or CXCL10). Without being bound by any particular theory of operation, it is believed that IL-12 through its ability to induce immune responses and its anti-angiogenic activity can be used to treat cancers.

Short circulatory half-life represents a major obstacle for many biologics, including cytokine based therapies. See, e.g., Perdreau et al., European Cytokine Network 21: 297-307 (2010). Such short-acting therapeutics require frequent dosing profiles that can reduce applicability to the clinic, particular for chronic conditions. Cytokines in particular, have been shown to be highly toxic when repeatedly administered. See, e.g., van der Poll et al., Cytokines as Regulators of Coagulation, Madame Curie Bioscience Database 2000. Long serum half-life is desirable as it would decrease the need for frequent injections of the molecule to achieve a therapeutically relevant serum concentration and low enough doses to be tolerable for patients.

Cytokine circulatory half-life can be extended by conjugating the cytokine to an albumin binding domain (ABD). Albumin binding domain (ABD) fusion proteins are shown to be useful for extending the half-lives of biologics (e.g., interleukins and antibodies). Serum albumin possess a long half-life in the range of 2-4 weeks due to recycling through the neonatal Fc receptor (FcRn). Albumin is taken up by endothelial cells through macropinocytosis and binds to the FcRn in a pH-dependent manner in the acidic environment of the early endosome. Albumin-FcRn binding diverts albumin molecules from degradation in the lysosomal compartment and redirects the albumin molecules to the plasma membrane, where they are released back into the blood plasma due to the neutral pH. Albumin binding domains (ABDs) do not compete with FcRn for albumin binding and bind albumin at a pH range that allows for the ABD to also undergo FcRn-driven endosomal albumin recycling when bound to albumin. As such, cytokine fusion proteins that include such albumin binding domain (ABD) are capable of evading lysosomal degradation using the albumin-FcRn pathway and, consequently, exhibit longer serum half-lives than counterparts lacking ABDs.

While cytokine based biologics, such as IL-12, have therapeutic potential, a challenge remains in eliminating proteolytic enzymes and protein aggregation during the manufacturing of such biologics. Interleukins are known for secreting proteolytic enzymes and causing aggregation in manufacturing. The proteolytic impact is that the cell culture contaminants and the proteolytic enzymes secreted naturally by the cytokine into the media, cause clipping or degradation of the intact molecule. Thus, there remains a need for improved methods for manufacturing cytokine based molecules, including IL-12 based therapies.

### SUMMARY

Provided herein are methods for making IL-12 albumin binding domain fusion proteins. The subject methods advantageously include a continuous downstream purification step that leads to high yield production of purified IL-12 albumin binding domain fusion proteins.

In one aspect, provided herein is a method of making an IL-12 albumin binding domain (ABD) fusion protein comprising: a) culturing a plurality of mammalian host cells that each comprise a polynucleotide encoding for the protein in a bioreactor comprising a liquid culture medium, wherein the host cells are cultured under conditions wherein the protein is produced and secreted by the host cells into the liquid culture medium; b) removing liquid culture medium comprising the secreted protein from the bioreactor; c) purifying the protein by passing the liquid culture medium removed from the bioreactor directly over one or more chromatography columns, wherein the one or more chromatography columns are operably and continuously linked to the perfusion bioreactor; and d) collecting the purified protein from the liquid culture medium.

In some embodiments, the bioreactor is a perfusion mode bioreactor. In some embodiments, the liquid culture medium comprising the secreted protein is removed from the perfusion mode bioreactor and replaced with fresh liquid culture medium at set time intervals. In exemplary embodiments, the set time interval is 12 hours, 24 hours, 48 hours, or 72 hours.

In some embodiments, the liquid culture medium comprising the secreted protein is continuously removed from the perfusion mode bioreactor and continuously replaced with fresh liquid culture medium.

In some embodiments, the liquid culture medium is removed from the bioreactor starting at least about 96 hours after beginning the culturing step a). In certain embodiments, the liquid culture medium comprising the secreted protein is removed from the perfusion mode bioreactor and replaced with an equal volume of fresh liquid culture medium. In some embodiments, a partial volume of the liquid culture medium comprising the secreted protein is partially removed from the perfusion mode bioreactor and replaced with an equal volume of fresh liquid culture medium.

In certain embodiments, the host cells are prevented from leaving the perfusion bioreactor during the removing step b) using a cell separation system.

In some embodiments, the host cells are cultured in the presence of nutrients that are periodically replenished in the liquid culture medium.

In some embodiments, the plurality of mammalian host cells are cultured at a density of 20-100×10⁶ cells/mL of liquid culture medium in the bioreactor.

In some embodiments, the purifying c) separates one or more proteolytic enzymes from the protein.

In some embodiments, the albumin binding domain of the IL-12 ABD fusion protein comprises a variable heavy domain comprising SEQ ID NO:1 or a variant thereof, and a variable light domain comprising SEQ ID NO:5 or a variant thereof. In certain embodiments, the albumin binding domain is an scFv that comprises the amino acid sequence of SEQ ID NO:9 or a variant thereof.

In some embodiments, the IL-12 of the IL-12 fusion protein is a single chain IL-12 comprising a p35 subunit covalently attached to a p40 subunit. In exemplary embodiments, the p40 subunit has the amino acid sequence of SEQ ID NO:10 or a variant thereof. In some embodiments, the p35 subunit has the amino acid sequence of SEQ ID NO:11 or a variant thereof. In some embodiments, the single chain IL-12 has the amino acid sequence of SEQ ID NO:12. In some embodiments, the IL-12 ABD fusion protein has the amino acid sequence of SEQ ID NO: 13.

In some embodiments, the one or more chromatography columns comprise an ion exchange chromatography column, a hydrophobic interaction column, an affinity column, a pseudo-affinity column or a size exclusion chromatography column. In exemplary embodiments, the one or more chromatography columns comprises an affinity column. In some embodiments, the pseudo-affinity column comprises a Cibacron Blue ligand. In exemplary embodiments, the pseudo-affinity column comprises an affinity chromatography media depicted in FIG. 14.

### BRIEF DESCRIPTION OF THE DRAWINGS

FIG. 1 provides a schematic of an exemplary embodiment of the subject manufacturing method provided herein. Host cells are grown in a standard stirred tank bioreactor. Perfusion is achieved by continuous feeding of fresh media. ATF device continuously removes the IL-12 albumin binding domain fusion protein product along with spent media. The perfusate is passed over capture columns, which are operated, in parallel mode wherein the collection alternates between the two columns (Column 1 and 2) to achieve continuous capture of the IL-12 albumin binding domain fusion protein product. The product can be eluted from the column, for example, by using conduction such as high conductivity or pH, which lead to the dissociation of the bound product.

FIG. 2 depicts a schematic of an exemplary embodiment of the upstream process of the subject manufacturing method provided herein. In such embodiments, cells (e.g. CHO cells) are initially grown in shake flasks and inoculated into a perfusion bioreactor at an inoculum viable cell density range of 1.2 to 1.8×10⁶ cells/mL, with target seeding density of 1.5×10⁶ cells/mL. In some embodiments, perfusion rate is started at 0.5 RV and increased as culture time and VCD rises. In some embodiments, the bioreactor is enabled with an alternating tangential flow (ATF) filter to allow collection of recombinant protein of interest (e.g., IL-12 albumin binding domain fusion protein) secreted into the culture medium. In some embodiments, the collected protein undergoes purification using an inline chromatography system that includes one or more different chromatography columns.

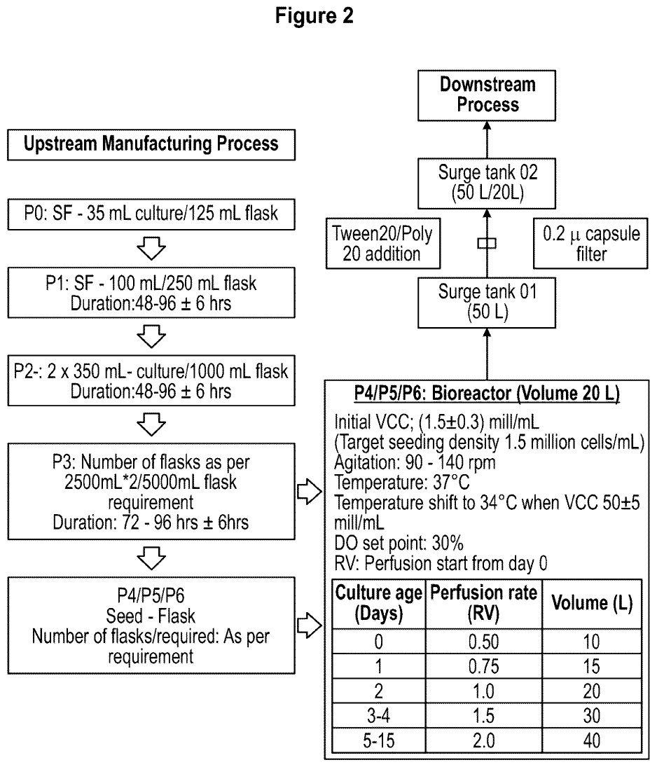

FIG. 3 depicts a schematic of an exemplary embodiment of the downstream process of the subject manufacturing method provided herein. In such embodiments, culture medium containing the protein of interest (e.g., IL-12 albumin binding domain fusion protein) collected from a continuous perfusion process is subjected to a chromatography system for isolation and purification of the protein of interest. In some embodiments, the protein of interest undergoes two or more chromatography processes. In exemplary embodiments, the protein of interest is subjected to affinity chromatography. In further embodiments, the protein of interest further undergoes hydrophobic interaction (HIC) chromatography and/or cation exchange (CEX) chromatography. Following chromatography purification, the protein of interest may further undergo a diafiltration/ultrafiltration and/or nanofiltration step.

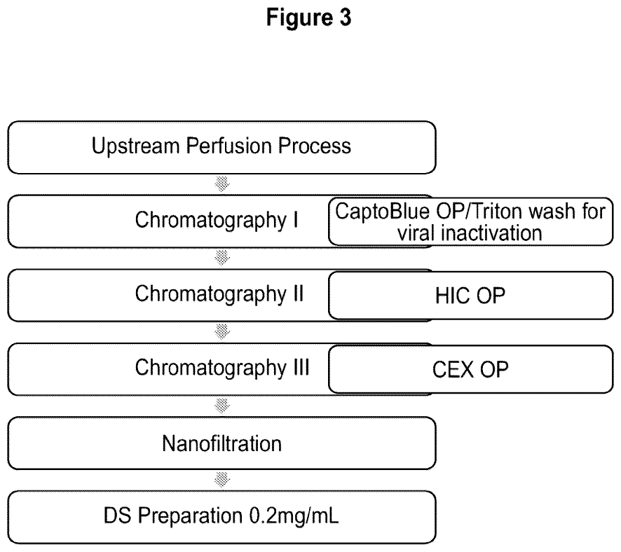

FIG. 4 depicts the albumin binding domain component of the IL-12 albumin binding domain fusion proteins that can be manufactured using the subject methods provided herein.

FIG. 5 depicts an exemplary IL-12 albumin binding domain (ABD) fusion protein that can be manufactured using the subject methods provided herein. The IL-12 depicted is a single chain IL-12 molecule, wherein the IL-12A (p35) and IL-12B (p40) components are attached using a polypeptide linker.

FIG. 6 depicts an assessment of viable cell densities and cell viability (A) and residual glucose (B), during the production of IL-12 ABD fusion protein using the subject continuous perfusion culturing method described herein (20 L). The parameters were assessed from aliquots of the culture medium taken every 24 hours.

FIG. 7 depicts an assessment of lactate profile (A) and glutamine profile (B), during the production of IL-12 ABD fusion protein using the subject continuous perfusion culturing method described herein (20 L). The parameters were assessed from aliquots of the culture medium taken every 24 hours.

FIG. 8 depicts an assessment of ammonia profile (A) and glutamine profile (B), during the production of IL-12 ABD fusion protein using the subject continuous perfusion culturing method described herein (20 L). The parameters were assessed from aliquots of the culture medium taken every 24 hours.

FIG. 9 provides an exemplary operating process flow for manufacturing scale production of IL-12 ABD fusion protein using the subject methods provided herein.

FIG. 10 depicts an affinity chromatography profile for a cycle of manufacturing scale production of IL-12/ABD fusion protein using the subject methods provided herein. The affinity column includes Capto™Blue medium for the capture of albumin.

FIG. 11 depicts the average loading density (mg/mL of resin) and recovery of protein of interest during the course of an exemplary chromatography purification step in production of IL-12/ABD fusion protein using the subject methods provided herein.

FIG. 12 depicts a representative reverse phase-high performance liquid chromatography (RP-HPLC) profile of a Capto™Blue affinity chromatography purification step during the production of IL-12/ABD fusion protein using the subject methods provided herein.

FIG. 13 depicts a representative reverse phase-high performance liquid chromatography (RP-HPLC) profile of a Capto™Blue affinity chromatography purification step during the production of IL-12/IL-15 ABD fusion protein (SON1210) using the subject methods provided herein.

FIG. 14 depicts exemplary pseudoaffinity ligands that include Cibacron Blue and can be used for the purification of the IL-12/ABD fusion proteins described herein.

### DETAILED DESCRIPTION

### I. Overview

Provided herein are methods for making IL-12 albumin binding domain fusion protein. In embodiments of the methods provided herein, the IL-12 albumin binding domain fusion protein is made in a bioreactor that is connected directly downstream to a purification system for purification. In embodiments, the purification system includes one or more chromatography columns for purification. In embodiments, after the protein is made and secreted into the liquid culture media in the bioreactor, the liquid culture media containing the protein is directly passed over to the purification system (e.g., one or more columns for purification) and the resulting purified product is collected. As the purification system (e.g., one or more chromatography columns) is directly and operatively linked to the bioreactor, the protein is passed onto the purification step from the bioreactor in a continuous manner, thereby minimizing the contact time with proteolytic enzymes secreted during the production process that can cause clipping and degradation. In contrast, previous cytokine production methods often include a holding step in which the protein is held for periods of times prior to purification, which increases exposure time to such proteolytic enzymes and leads to protein degradation. As such, the subject methods advantageously allow for high yield production of IL-12 albumin binding domain fusion proteins that exhibit less degradation than those produced using previous methods. Aspects of the subject methods are described in greater detail.

### II. Definitions

In order that the application may be more completely understood, several definitions are set forth below. Such definitions are meant to encompass grammatical equivalents.

The term “a” or “an” refers to one or more of that entity, i.e. can refer to a plural referent. As such, the terms “a” or “an”, “one or more” and “at least one” are used interchangeably herein. In addition, reference to “an element” by the indefinite article “a” or “an” does not exclude the possibility that more than one of the elements is present, unless the context clearly requires that there is one and only one of the elements.

It is noted that the claims may be drafted to exclude any optional element. As such, this statement is intended to serve as an antecedent basis for use of such exclusive terminology as “solely,” “only,” and the like in connection with the recitation of claim elements or use of a “negative” limitation. As will be apparent to those of skill in the art upon reading this disclosure, each of the individual embodiments described and illustrated herein has discrete components and features readily separated from or combined with the features of any of the other several embodiments without departing from the scope or spirit of the invention. Any recited method may be carried out in the order of events recited or in any other order that is logically possible. Although any methods and materials similar or equivalent to those described herein may also be used in the practice or testing of the invention, representative illustrative methods and materials are now described.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

Unless specifically stated or obvious from context, as used herein, the term “about” is understood as within a range of normal tolerance in the art, for example within 2 standard deviations of the mean. About can be understood as within 50%, 45%, 40%, 35%, 30%, 25%, 20%, 15%, 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Before the invention is further described, it is to be understood that this invention is not limited to particular embodiments described, as such may, of course, vary. It is also to be understood that the terminology used herein is for describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are encompassed within the invention, subject to any specifically excluded limit in the stated range. Where the stated range includes one or both of the limits, ranges excluding either or both of those included limits are also included in the invention. Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number, which, in the context presented, provides the substantial equivalent of the specifically recited number.

All publications, patents, and patent applications cited in this specification are incorporated herein by reference to the same extent as if each individual publication, patent, or patent application were specifically and individually indicated to be incorporated by reference. Furthermore, each cited publication, patent, or patent application is incorporated herein by reference to disclose and describe the subject matter in connection with which the publications are cited. The citation of any publication is for its disclosure prior to the filing date and should not be construed as an admission that the invention described herein is not entitled to antedate such publication by virtue of prior invention. Further, the dates of publication provided might be different from the actual publication dates, which may need to be independently confirmed.

### III. IL-12 Albumin Binding Domain Fusion Protein

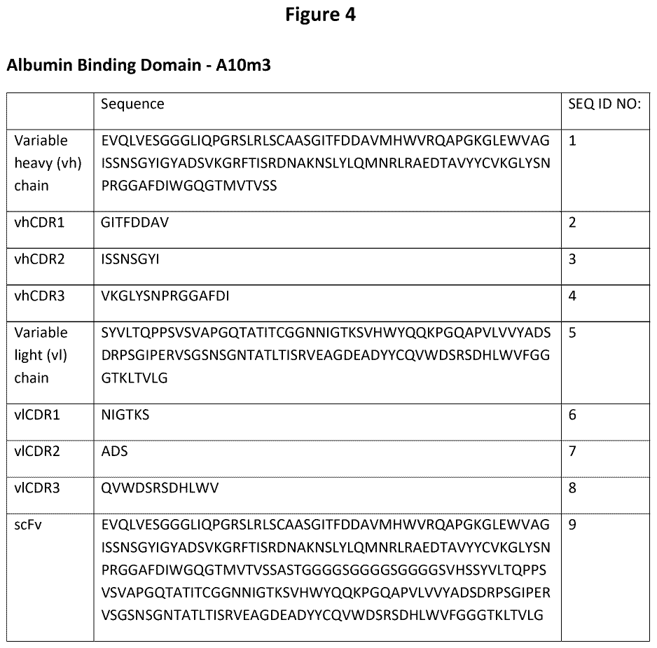

In embodiments, the subject methods described herein are useful for producing IL-12 albumin binding domain (ABD) fusion proteins. Such IL-12 ABD fusion protein find use, for example, in the treatment of cancers. In some embodiments, the IL-12 albumin binding domain fusion protein includes an albumin binding domain that includes an antibody variable heavy chain domain that includes a vhCDR1 having the amino acid sequence of SEQ ID NO:2, a vhCDR2 having the amino acid sequence of SEQ ID NO:3, and a vhCDR3 having the amino acid sequence of SEQ ID NO:4 (see FIG. 4). In some embodiments, the albumin binding domain includes a variable light chain domain that includes a vlCDR1 having the amino acid sequence of SEQ ID NO:6, a vlCDR2 having the amino acid sequence of SEQ ID NO:7, and a vlCDR3 having the amino acid sequence of SEQ ID NO:8. In embodiments, the albumin binding domain includes the variable heavy chain having the amino acid sequence of SEQ ID NO:1 and/or a variable light chain having the amino acid sequence of SEQ ID NO:5. In embodiments, the albumin binding domain includes the variable heavy chain having the amino acid sequence of SEQ ID NO:1 and a variable light chain having the amino acid sequence of SEQ ID NO:5. In embodiments, the albumin binding domain of the IL-12 albumin binding domain fusion protein is an scFv that has the amino acid sequence of the A10m3 (SEQ ID NO:9).

In embodiments, the IL-12 albumin binding domain fusion protein includes an albumin binding domain that is a variant of the A10m3 albumin binding domain depicted in FIG. 4. In exemplary embodiments, the albumin binding domain of the IL-12 albumin binding domain fusion protein includes a set of 6 CDRs with 1, 2, 3, 4, 5, 6, 7, 8, 9, 10 amino acid modifications as compared to the 6 CDRs of A10m3, as depicted in FIG. 4. In embodiments, the albumin binding domain of the IL-12 albumin binding domain fusion protein includes 6 CDRs that are at least 90, 95, 97, 98 or 99% identical to the 6 CDRs of A10m3 (see FIG. 4). In embodiments, the albumin binding domain includes a VH domain and/or VL domain that has from 1, 2, 3, 4, 5, 6, 7, 8, 9 or 10 amino acid changes from a VH and/or VL domain of A10m3, as depicted in FIG. 4. In embodiments, the albumin binding domain includes a VH domain and/or VL domain that is at least 90, 95, 97, 98 or 99% identical to the VH and/or VL of A10m3, as depicted in FIG. 4. In certain embodiments, the variant of the A10m3 albumin binding domain is capable of binding human serum albumin, as measured by at least one of a Biacore, surface plasmon resonance (SPR), flow cytometry, and/or BLI (biolayer interferometry, e.g., Octet assay) assay, with the latter finding particular use in many embodiments.

In certain embodiments, the IL-12 is a single chain IL-12 polypeptide comprising an IL-12 p35 subunit attached to an IL-12 p40 subunit. In embodiments, the IL-12 single chain polypeptides advantageously retain one or more of the biological activities of wildtype IL-12. In some embodiments, the single chain IL-12 polypeptide described herein is according to the formula, from N-terminus to C-terminus, (p40)-(L)-(p35), wherein “p40” is an IL-12 p40 subunit, “p35” is IL-12 p35 subunit and L is a linker. In other embodiments, the single chain IL-12 is according to the formula from N-terminus to C-terminus, (p35)-(L)-(p40). Any suitable linker can be used in the single chain IL-12 polypeptide. Suitable linkers can include, for example, linkers having the amino acid sequence (GGGGS)ₓ wherein x is an integer from 1-10. Other suitable linkers include, for example, the amino acid sequence GGGGGGS. Exemplary single chain IL-12 linkers than can be used with the subject single chain IL-12 polypeptides are also described in Lieschke et al., Nature Biotechnology 15: 35-40 (1997), which is incorporated herein in its entirety by reference and particularly for its teaching of IL-12 polypeptide linkers.

In some embodiments, the IL-12 of the IL-12 albumin binding domain fusion protein includes a human p40 subunit having the amino acid sequence of SEQ ID NO:10. In some embodiments, the IL-12 includes a variant human p40 subunit that has from 1, 2, 3, 4, 5, 6, 7, 8, 9 or 10 amino acid changes as compared to SEQ ID NO:10. In embodiments, the IL-12 includes a variant human p40 that is at least 90, 95, 97, 98 or 99% identical to SEQ ID NO:10.

In some embodiments, the IL-12 of the IL-12 albumin binding domain fusion protein includes a human p35 subunit having the amino acid sequence of SEQ ID NO:11. In some embodiments, the IL-12 includes a variant human p35 subunit that has from 1, 2, 3, 4, 5, 6, 7, 8, 9 or 10 amino acid changes as compared to SEQ ID NO:10. In embodiments, the IL-12 includes a variant human p40 that is at least 90, 95, 97, 98 or 99% identical to SEQ ID NO:11.

### IV. Cell Culture

In some embodiments of the subject method, the IL-12 albumin binding domain fusion protein is made in a bioreactor by culturing mammalian host cells that include a polynucleotide encoding the protein in the bioreactor. In embodiments, the host cells are cultured in a liquid culture media under conditions where the protein is produced and secreted by the host cell into the liquid culture media.

Any suitable host cell can be used for the production of the IL-12 albumin binding domain fusion protein. In exemplary embodiments, the host cell is a mammalian host cell. A wide variety of mammalian cell lines suitable for growth in culture are available from the American Type Culture Collection (Manassas, Va.) and commercial vendors. Examples of cells that can be used with the subject methods include, but are not limited to, VERO, BHK, HeLa, CV1 (including Cos), MDCK, 293, 3T3, myeloma cell lines (e.g., NSO, NSl), PC12, WI38 cells, and Chinese hamster ovary (CHO) cells. CHO cells are widely used for the production of complex recombinant proteins, e.g. cytokines, clotting factors, and antibodies (Brasel et al. (1996), Blood 88:2004-2012; Kaufman et al. (1988), J. Biol Chem 263:6352-6362; McKinnon et al. (1991), J Mol Endocrinol 6:231-239; Wood et al. (1990), J. Immunol. 145:3011-3016). The dihydrofolate reductase (DHFR)-deficient mutant cell lines (Urlaub et al. (1980), Proc Natl Acad Sci USA 77: 4216-4220), DXB11 and DG-44, and CHO-K1, are desirable CHO host cell lines because the efficient DHFR selectable and amplifiable gene expression system allows high level recombinant protein expression in these cells (Kaufman RJ. (1990), Meth Enzymol 185:537-566). In addition, these cells are easy to manipulate as adherent or suspension cultures and exhibit relatively good genetic stability. CHO cells and proteins recombinantly expressed in them have been extensively characterized and have been approved for use in clinical commercial manufacturing by regulatory agencies.

The host cell expressing the recombinant IL-12 albumin binding domain fusion protein can be cultured in any suitable medium that allows for growth of the host cell and expression of the recombinant protein. Cell culture media formulations are well known in the art. Typically, cell culture media are comprised of buffers, salts, carbohydrates, amino acids, vitamins and trace essential elements. The cell culture medium may or may not contain serum, peptone, and/or proteins. Various cell culture media, including serum-free and defined culture media, are commercially available, for example, any one or a combination of the following cell culture media can be used: RPMI-1640 Medium, RPMI-1641 Medium, Dulbecco's Modified Eagle's Medium (DMEM), Sartorius Stedim Cellca's smd (CHOKO), Cellca FMA, Cellca FMB (SAFC), Acti-Pro (GE), Minimum Essential Medium Eagle, F-12K Medium, Ham's F12 Medium, Iscove's Modified Dulbecco's Medium, McCoy's 5A Medium, Leibovitz's L-15 Medium, and serum-free media such as EX-CELL™ 300 Series (JRH Biosciences, Lenexa, Kansas), among others. Cell culture media may be supplemented with additional or increased concentrations of components such as amino acids, salts, sugars, vitamins, hormones, growth factors, buffers, antibiotics, lipids, trace elements and the like, depending on the requirements of the cells to be cultured and/or the desired cell culture parameters. Cell culture media may be serum-free, protein-free, and/or peptone-free. “Serum-free” applies to a cell culture medium that does not contain animal sera, such as fetal bovine serum. “Protein-free” applies to cell culture media free from exogenously added protein, such as transferrin, protein growth factors IGF-1, or insulin. Protein-free media may or may not contain peptones. “Peptone-free” applies to cell culture media which contains no exogenous protein hydrolysates such as animal and/or plant protein hydrolysates. Eliminating serum and/or hydrolysates from cell culture media has the advantage of reducing lot to lot variability and enhancing processing steps, such as filtration. However, when serum and/or peptone are removed from the cell culture media, cell growth, viability and/or protein expression may be diminished or less than optimal. As such, serum-free and/or peptone-free cell culture medium may be highly enriched for amino acids, trace elements and the like. See, for example, U.S. Pat. Nos. 5,122,469 and 5,633,162. Defined cell culture media formulations are complex, containing amino acids, inorganic salts, carbohydrates, lipids, vitamins, buffers and trace essential elements. Components that are necessary and beneficial to maintain a cell culture with desired characteristics will depend on the particular host cell used and the manner in which the bioreactor is operated.

The host cells may be cultured using any suitable technique that allows for the growth of the cell and expression of the IL-12 albumin binding domain fusion protein. Mammalian cells may be cultured in suspension or while attached to a solid substrate. In some embodiments, the mammalian cells are cultured in a bioreactor. Exemplary bioreactors include, but are not limited to fluidized bed bioreactors, hollow fiber bioreactors, roller bottles, shake flasks, or stirred tank bioreactors, with or without microcarriers. Bioreactors can be operated in a batch, fed batch, continuous, semi-continuous, or perfusion mode. In particular embodiments, the culturing is a large scale culture where the culturing is carried out in a volume of at least about 10 L, at least about 20 L, at least about 25 L, at least about 50 L, at least about 75 L, at least about 100 L, at least about 500 L, at least about 1000 L, at least about 2000 L, at least about 3000 L, at least about 5,000 L, at least about 7,000 L, at least about 8,000 L, at least about 10,000 L, at least about 15,000 L, or at least about 20,000 L of culture media. In some embodiments, the culturing step is carried out in a volume of 30 mL-50 L of culture media. In embodiments, the culturing step is carried out at 1 L-10 L, 10 L-20 L, 20 L-50 L, 50 L-100 L of culture medium.

In embodiments, the mammalian cells are cultured in a fed batch mode. Fed batch mode refers to a culture of mammalian cells is one in which the culture is fed, either continuously or periodically, with a concentrated feed medium containing nutrients. In embodiments, feeding occurs on a predetermined schedule of, for example, every day, once every two days, once every three days, etc. The culture can be monitored for tyrosine, cystine and/or cysteine levels in the culture medium and can be adjusted through feedings of a concentrated tyrosine or tyrosine and cystine solution so as to keep tyrosine, cysteine and/or cystine within a desired range. When compared to a batch culture, in which no feeding occurs, a fed batch culture can produce greater amounts of protein. In embodiments, the mammalian cells are cultured in a continuous fed batch mode. In embodiments, the mammalian cells are cultured in a periodic fed batch mode.

In exemplary embodiments, the bioreactor is operated in a perfusion mode. In a perfusion mode, the IL-12 albumin binding domain fusion protein is regularly removed from the bioreactor and replaced with fresh media. As such, the host cells are not exposed to increasing concentrations of toxic byproducts (e.g., proteolytic enzymes) generated during the cell culture process, thereby minimizing degradation. In some embodiments, the IL-12 albumin binding domain fusion protein is continuously removed together with media and replaced with an equal volume of fresh media. In some certain embodiments, the IL-12 albumin binding domain fusion protein is removed together with media at fixed regular time intervals of time and replaced with an equal volume of fresh media. In some embodiments, the time interval is about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, or about 24 hours. In some embodiments, the time interval is about 1, about 2, about 3, about 4, about 5, about 6, about 7, about 8, about 9, or about 10 or more days.

In embodiments under perfusion conditions, the cell culture can proceed for many weeks to months. In some embodiments, the bioreactor is operated under perfusion conditions for about 1, about 2, about 3, about 4, about 5, about 6, or about 7 days. In certain embodiments, the bioreactor is operated under perfusion condition for 1, 2, 3, or 4 weeks. In some embodiments, the bioreactor is operated under perfusion conditions up to about 1, about 2, about 3, about 4, about 5, about 6, about 7, about 8, about 9, about 10 or about 12 months.

In embodiments, the cell culture is maintained at about 20° C.-about 50° C. In embodiments, the reaction is performed at about 25° C.-about 45° C. In some embodiments, the reaction is performed at about 30° C.-about 40° C. In some embodiments, the temperature of the cell culture is maintained at the same temperature when the cells are in growth phase and production phase. In embodiments, the temperature of the cell culture is within about 1° C., 2° C., 3° C., 4° C., 5° C., 6° C., 7° C., 8° C., 9° C., or 10° C. during growth phase and production phase.

In embodiments, the pH of the cell culture is maintained at a pH of about 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0. In embodiments, the pH of the cell culture is maintained at about 6.0-about 8.0. In some embodiments, the pH of the cell culture is maintained at about 6.5-7.5.

In exemplary embodiments, the bioreactor is operated under perfusion conditions wherein the host cells are allowed to grow at high densities. In particular embodiments, the host cells are grown to a density of at least 10×10⁶ cells/mL, 15×10⁶ cells/mL, 25×10⁶ cells/mL, 30×10⁶ cells/mL, 35×10⁶ cells/mL, 40×10⁶ cells/mL, 45×10⁶ cells/mL, 50×10⁶ cells/mL, 55×10⁶ cells/mL, 60×10⁶ cells/mL, 70×10⁶ cells/mL, 80×10⁶ cells/mL, 90×10⁶ cells/mL or 100×10⁶ cells/mL. Growing the cells at a high density allow the production of more IL-12 albumin binding domain fusion protein in the same volume of media over a standard perfusion process.

In embodiments, the host cells are initially inoculated in a bioreactor at a cell density of from about 0.5×10⁶ cells/mL-about 3.0×10⁶ cells/mL. In embodiments, the host cells are initially inoculated in a bioreactor at a cell density of from about 1×10⁶ cells/mL to about 2.0×10⁶ cells/mL. In certain embodiments, the host cells are allowed to go through multiple growth phases to maximize protein production. In exemplary embodiments, the host cells go through at least 2, 3, 4, 5, 6, 7, 8, 9, 10 grown phases in the bioreactor before the final production phase.

### V. Purification

In embodiments of the subject method, culture media containing the IL-12 albumin binding domain fusion proteins produced during the cell culturing step are removed from the bioreactor and purified by directly subjecting the culture media containing the IL-12 albumin binding domain fusion proteins to a purification system. The resulting purified product is collected. In embodiments, the purification system includes one or more chromatography columns. As the purification system (e.g., one or more chromatography columns) are directly and operatively linked to the bioreactor, the IL-12 albumin binding domain fusion proteins are passed onto the purification step from the bioreactor in a continuous manner, thereby minimizing the contact time with proteolytic enzymes secreted by the cytokine that can cause clipping and degradation. In contrast, previous cytokine production methods often include a holding step in which the protein is held for periods of times prior to purification, which increases exposure time to such proteolytic enzymes and leads to protein degradation. As such, the subject methods advantageously allow for high yield production of cytokines-based products that exhibit less degradation than those produced using previous methods.

In some embodiments, viable host cells, as well as cellular debris, are prevented from leaving the bioreactor with the culture media containing the protein product. Any suitable technique can be used to prevent the host cells from leaving the bioreactor. In particular embodiments a filtration system is used keep the host cells from being removed along with the culture media. Any suitable filtration system that includes a membrane that retains cells and allows the IL-12 ABD product to go through the membrane can be used. In exemplary embodiments, the filtration system is an ATF or TFF filtration system. In some embodiments the cells are kept in the bioreactor using gravity settling, pumping through internal filters, external loop flow-through filters and centrifugation techniques.

‘In embodiments, culture medium containing the IL-12 ABD product is subjected to the purification system for purification at least about 12 hours, at least about 18 hours, at least about 24 hours at least about 30 hours, at least about 36 hours, at least about 42 hours, at least about 48 hours, at least about 54 hours, at least about 60 hours, at least about 66 hours, at least about 72 hours, at least about 78 hours, at least about 84 hours, at least about 90 hours, at least about 96 hours, at least about 102 hours, at least about 108 hours, at least about 114 hours, at least about 120 hours, at least about 126 hours, at least about 132 hours, at least about 144 hours after the start of the culture medium.

In embodiments, the purification step occurs for at least about 6 hours, at least about 12 hours, at least about 18 hours, at least about 24 hours, at least about 2 days, at least about 3 days, at least about 4 days, at least about 5 days, at least about 6 days, at least about 7 days, at least about 9 days, at least about 10 days, at least about 11 days, at least about 12 days, at least about 13 days, at least about 14 days, at least about 3 weeks, or a month. In some embodiments, the culture medium containing the IL-12 ABD product is subjected to the purification system for purification if the cell viability of the culture medium is at least about 60%, at least about 65%, at least about 70%, at least about 75%, at least about 80%, at least about 85%, at least about 90%, or at least about 95%.

Any suitable chromatography columns and combinations thereof can be used to purify the protein product from the spent media. Chromatography columns that can be used in the subject methods include, but are not limited to ion-exchange chromatography, size-exclusion chromatography, hydrophobic interaction chromatography, and affinity chromatography. In preferred embodiments, a first column is directly and operable linked to the bioreactor, thereby allowing for continuous downstream purification of the protein product from the spent media. In certain embodiments, additional columns are connected in stepwise fashion to the first column.

In certain embodiments, the culture media containing the protein product is subjected to at least one affinity chromatography column to further purify the protein product away from contaminants including proteolytic impurities. In affinity chromatography, a target molecule is separated from a complex solution based on the affinity of the target molecule for a ligand or ligand-binding entity that is covalently bound to the matrix. Molecules in the complex solution or mixture with weak affinity, or lacking affinity, for the ligand or ligand-binding entity flow through the chromatography column unimpeded, leaving the target molecule bound to the matrix. The target molecule can then be eluted from the chromatography column by altering buffer conditions to decrease the affinity of the target molecule for the ligand or ligand-binding entity. In certain embodiments the chromatographic material is capable of selectively or specifically binding to the protein of interest. Non-limiting examples of such chromatographic material include: Protein A, Protein G, Protein L, and chromatographic material comprising a molecule that binds the protein product. In some embodiments, the subject method utilizes affinity chromatography that includes a ligand or ligand binding entity capable of binding the albumin binding domain of the IL-12 ABD fusion protein. In some embodiments, the affinity chromatography includes an antibody that is capable of binding the albumin binding domain of the IL-12 ABD fusion protein. In some embodiments, the antibody of the chromatography column is capable of binding A10m3 albumin binding domain (see FIG. 4).

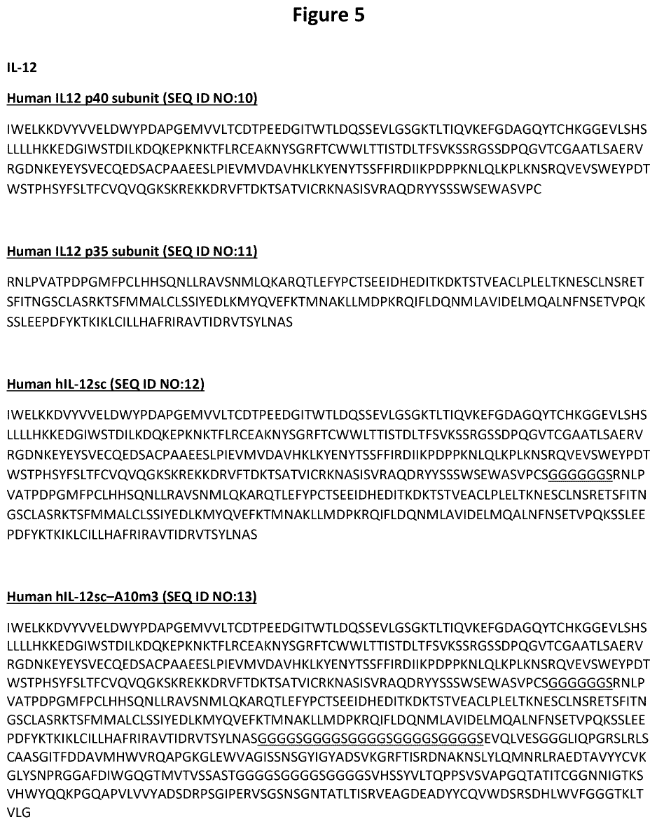

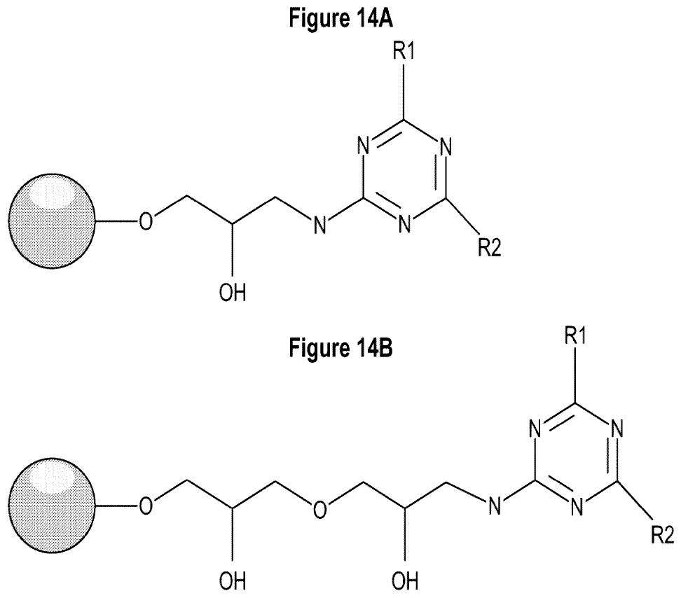

In some embodiments, the purification system includes a pseudo-affinity chromatography column. Pseudo-affinity columns utilizes dyes as ligands in order to bind proteins of interest. In some embodiments, the pseudo-affinity column includes a ligand capable of binding the IL-12 ABD fusion protein of interest (see FIG. 5). IL-12 ABD fusion protein is then subsequently captured by the ligand/serum albumin complex. In some embodiments, the pseudoaffinity column includes a Cibacron Blue ligand. In certain embodiments, the Cibacron Blue ligand is attached to an agarose base matrix by a hydrophilic spacer. In some embodiments, the pseudo-affinity column includes Capto™Blue resin (see FIG. 14). Capto™Blue resin includes a Cibacron Blue ligand attached to an agarose base matrix by a hydrophilic spacer.

In certain embodiments, the culture media containing the protein product is subjected to at least one ion exchange separation step such that an eluate comprising the protein product is obtained. Ion exchange separation includes any method by which two substances are separated based on the difference in their respective ionic charges, and can employ either cationic exchange material or anionic exchange material.

The use of a cationic exchange material versus an anionic exchange material is based on the overall charge of the protein. Therefore, it is within the scope of this invention to employ an anionic exchange step prior to the use of a cationic exchange step, or a cationic exchange step prior to the use of an anionic exchange step. Furthermore, it is within the scope of the subject methods to employ only a cationic exchange step, only an anionic exchange step, or any serial combination of the two.

In certain embodiments, the culture media containing the protein product is subjected to at least one size chromatography column. In size-exclusion chromatography, a target molecule is separated from a complex solution or mixture based on the target molecule's size-related exclusion from the interior regions of spherical beads that make up the matrix. Progress through the chromatography column of smaller molecules that are capable of diffusing into the beads is slowed with respect to the target molecule.

In certain embodiments, the culture media containing the protein product is subjected to a hydrophobic interaction (HIC) chromatography column. In hydrophobic interaction chromatography, a target molecule is separated from a complex solution or mixture based on the hydrophobicity of the target molecule. A complex solution containing the target molecule is applied to a chromatography column equilibrated with a high salt buffer that facilitates binding of the target molecule to the resin. A salt-gradient mobile phase with decreasing ionic strength is then introduced into the chromatography column to release bound target molecules from the matrix. Alternatively, hydrophobic interaction chromatography may separate a monomeric target molecule from a complex solution or mixture by binding hydrophobic impurities, including inactive dimers and aggregates of the target molecule, while permitting monomeric target molecules to flow through the chromatography column relatively unimpeded.

After purification of the protein product is collected, the protein product is tested for purity. Purity of the protein product can be tested using any suitable technique. In some embodiments, the purity of the protein product is determined using SEC-HPLC, CE-SDS Reduced, CE-IEF, Glycan Analysis by LC/MS or RP-HPLC. In exemplary embodiments, the protein product is of a purity that is great than 65%, 70%, 75%, 80%, 85%, 90%, 95% or 99%.

All cited references are herein expressly incorporated by reference in their entirety.

Whereas particular embodiments of the invention have been described above for purposes of illustration, it will be appreciated by those skilled in the art that numerous variations of the details may be made without departing from the invention as described in the appended claims.

### Examples

Examples are provided below to illustrate the present invention. These examples are not meant to constrain the present invention to any particular application or theory of operation.

### IL-12 Albumin Binding Domain (ABD) Fusion Protein Cell Culture Step

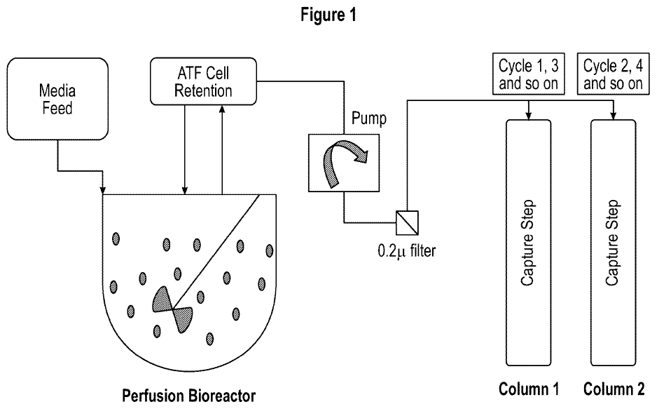

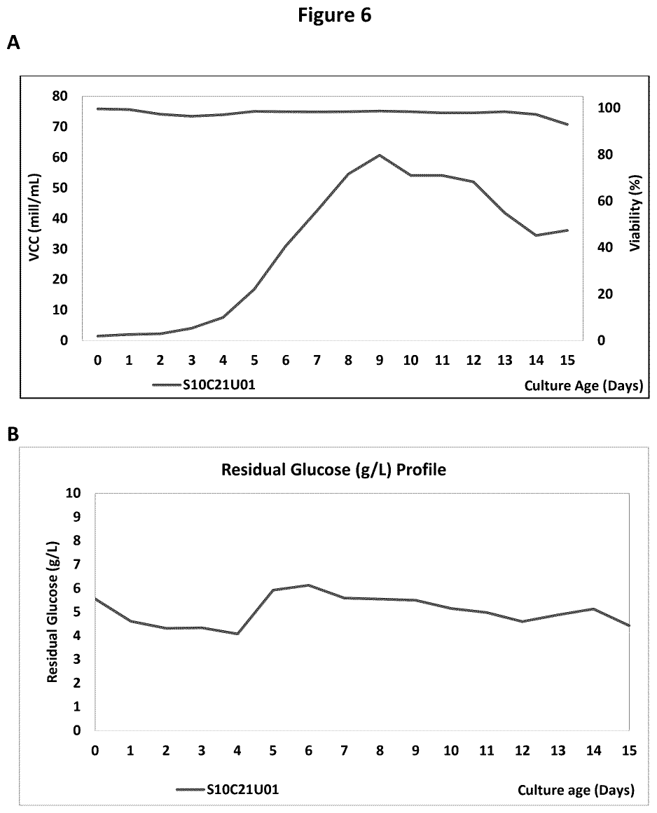

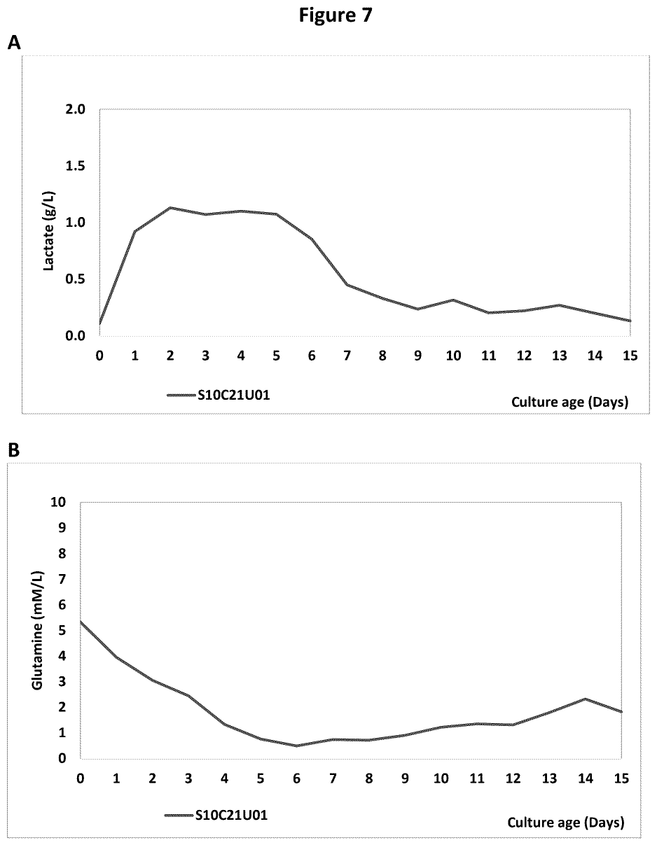

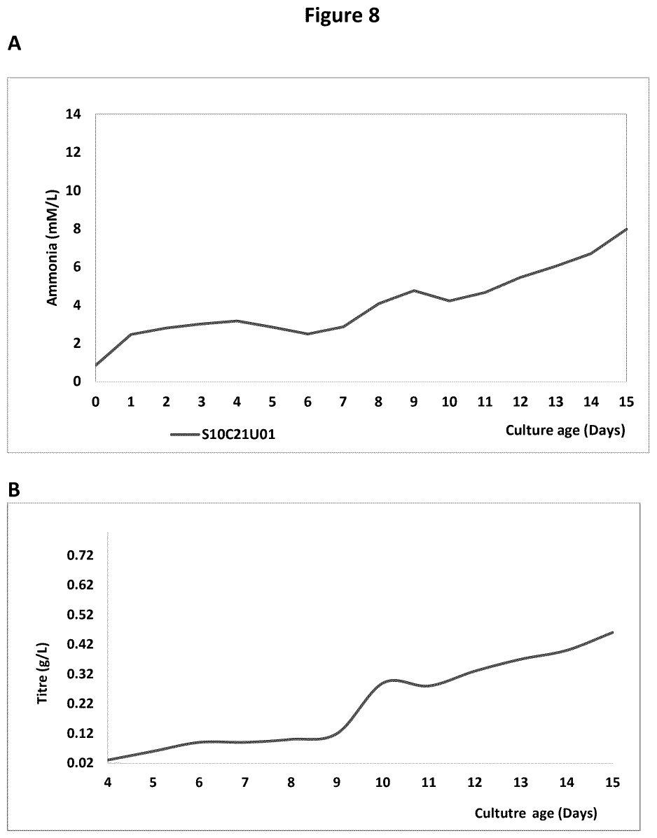

Production of an IL-12 albumin binding domain fusion protein (IL-12 ABD) was carried out using a continuous process. The IL-12 ABD includes an anti-albumin scFv termed “A10m3” that is linked to a single chain IL-12 (see FIG. 5). For the continuous process, a perfusion based system was used in which an alternating tangential flow (ATF) through a hollow fiber filter is used to establish a highly efficient filtering. This mechanism allows for the growth of cells up to densities of 20-100 million cells per milliliter with a viability of more than 85% for the duration of 25 days. The upstream batches were carried out at varying scales, ranging from 2.5 L to 20 L in manufacturing unit (GMP). At 2.5 L, perfusion runs were performed in a discontinuous fashion but processed immediately to simulate a continuous process, however at manufacturing scale, perfusion upstream was followed by an inline capture step from downstream. Bioreactors (Make: Sartorius, Germany) with cell retention device ATF 4 were loaded with the required working volume of cell culture media (XtraCHO) during batch manufacture at 2.5 L, and in Manufacturing (GMP), both ATF 4 as well as ATF 6 (Make: Repligen, USA) were used. The dissolved oxygen concentration was managed at 30 percent (range 20-50 percent) of air saturation by automatic modification of incoming gas composition through the headspace and intermittent sparging through a ring sparger across all scales. The pH set point was 7.0 (range 6.8-7.4) and controlled by the flow of CO2 via the sparger. Cells were inoculated in the bioreactor with an inoculum viable cell density range of 1.2 to 1.8×10⁶ cells/mL (FIG. 1) and with target seeding density of 1.5×10⁶ cells/mL. Perfusion rate (FIG. 1) was started at 0.5 RV of XtraCHO with feed medium A (FMA) and feed medium B (FMB) at 0 hr and increased as culture time and VCD rose (FIG. 1). The harvest from 0 hr to 96 hr was discarded, and the capture inline phase was started from 96 hr and continued till 120 hr (day 5), 144 hr (day 6), 168 hr (day 7), 192 hr (day 8), 216 hr (day 9), 240 hr (day 10) until 360 hr (day 15) as long as the viability was maintained more than 85%. Small aliquots of the culture were collected every 24 hours to check parameters such as VCD (FIG. 6A), viability (FIG. 6A), residual glucose (FIG. 6B), residual glutamine (FIG. 7B), lactate (FIG. 7A), ammonia levels (FIG. 8A), and titres (FIG. 8B). The bioreactor enabled with alternating tangential flow (ATF) filter to collect a harvest comprising the protein secreted into the culture medium; a first chromatography system connected to the surge bag of the bioreactor to purify the harvested recombinant therapeutic protein. The process parameters and the overview of volumetric and cumulative productivities are shown in Table 1 and Table 2.

TABLE 1
Process parameters at 2.5 L scale

| Process Parameters | Growth Phase | Production Phase |
|---|---|---|
| Temperature (° C.) | 37 ± 0.5 | 34 ± 0.5 |

| | |
|---|---|
| pH set point | 7.00 (Dead Band-0.2) |
| DO set point | 30% |
| Agitation (RPM) | 200 to 300 RPM |
| ATF 4 flow rate | 1.5 LPM-2.5 LPM |
| pH Range during Batch | 6.80 to 7.40 |
| Gassing Strategy | 0 to 1 LPM Air sparging |
| | 0 to 1 LPM Oxygen |
| | 0 to 1 LPM Air overlay |
| | 0 to 1 LPM CO2 Sparging. |
| | Air:Oxygen in 1:3 ratio to be maintained |

TABLE 2
Process parameters at 20 L scale

| Process Parameters | Growth Phase | Production Phase |
|---|---|---|
| Temperature (° C.) | 37 ± 0.5 | 34 ± 0.5 |

| | |
|---|---|
| pH set point | 7.00 (Dead Band-0.2) |
| DO set point | 30% |
| Agitation (RPM) | 90 to 160 RPM |
| ATF 4 flow rate | 3 LPM-8 LPM |
| ATF 6 flow rate | 10 LPM-20 LPM |
| pH Range during Batch | 6.80 to 7.40 |
| Gassing Strategy | 1 to 5 LPM Air sparging |
| | 1 to 5 LPM Oxygen |
| | 0 to 5 LPM Air overlay |
| | 0 to 5.0 LPM CO2 Sparging. |
| | Air:Oxygen in 1:3 ratio to be maintained |

### IL-12 Albumin Binding Domain (ABD) Fusion Protein Purification Step

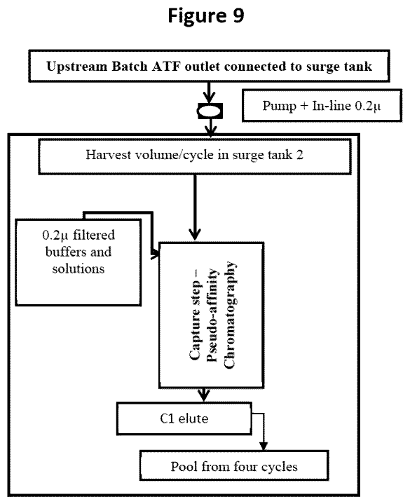

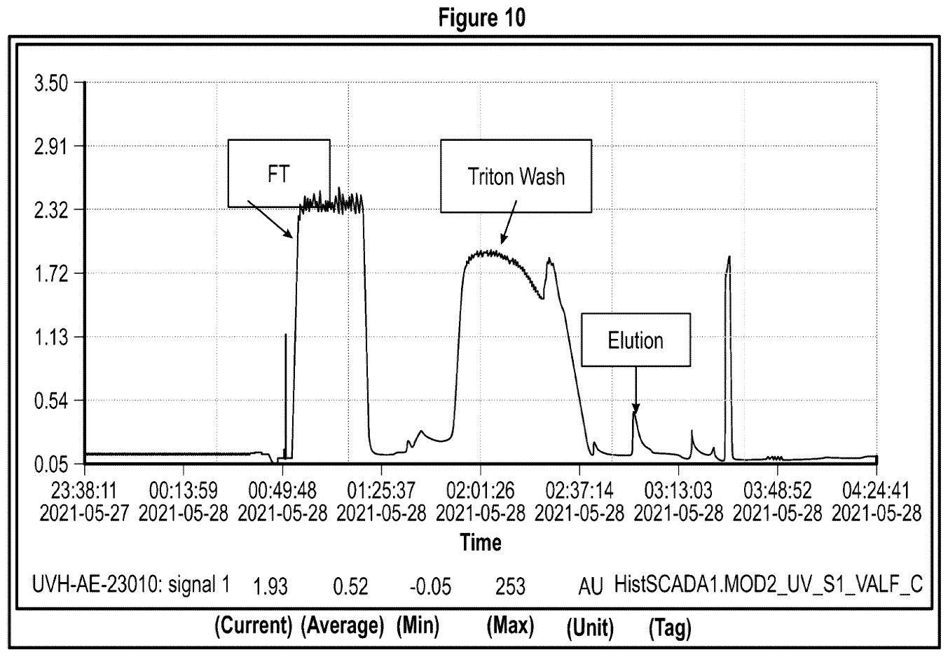

The IL12-ABD molecule was captured from the ATF output of the bioreactor by loading on an affinity-based chromatography column (Capto™Blue) connected to Novasep automated continuous chromatography system. The Capto™Blue resin was packed in the column and harvest was directly loaded onto the column without any external modification or treatment such as pH or dilution (FIG. 9). The loading factor were obtained in the range of 0.7-2.5 mg/mL of resin. The process can be operated in the range of 2.5-4 min residence time and was performed at a residence time of 4 min in case of batch taken at manufacturing scale. The on-column Triton X-100 wash was incorporated as a potential step for the viral inactivation in the chromatographic step (FIG. 10). The process execution parameters are as below (Table 3).

TABLE 3
Overview data of 20 L scale

| Culture | Harvest | Perfusion | Vol. | Cumulative |
|---|---|---|---|---|
| Age | Titer | Vol | Productivity | productivity |
| (Days) | (g/L) | in (L) | (g/Day) | (g) |
| 4 | 0.03 | 30 | 0.90 | 0.9 |
| 5 | 0.06 | 40 | 2.40 | 3.3 |
| 6 | 0.09 | 40 | 3.60 | 6.9 |
| 7 | 0.09 | 40 | 3.60 | 10.5 |
| 8 | 0.10 | 40 | 4.00 | 14.5 |
| 9 | 0.12 | 40 | 4.80 | 19.3 |
| 10 | 0.29 | 40 | 11.60 | 30.9 |
| 11 | 0.28 | 40 | 11.20 | 42.1 |
| 12 | 0.33 | 40 | 13.20 | 55.3 |
| 13 | 0.37 | 40 | 14.80 | 70.1 |
| 14 | 0.40 | 40 | 16.00 | 86.1 |
| 15 | 0.46 | 40 | 18.40 | 104.5 |

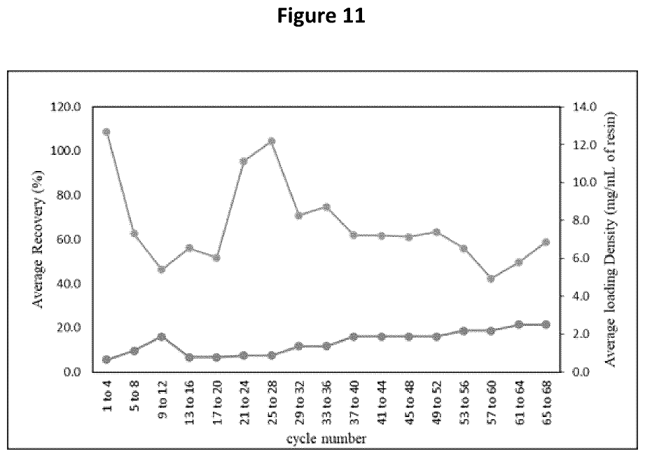

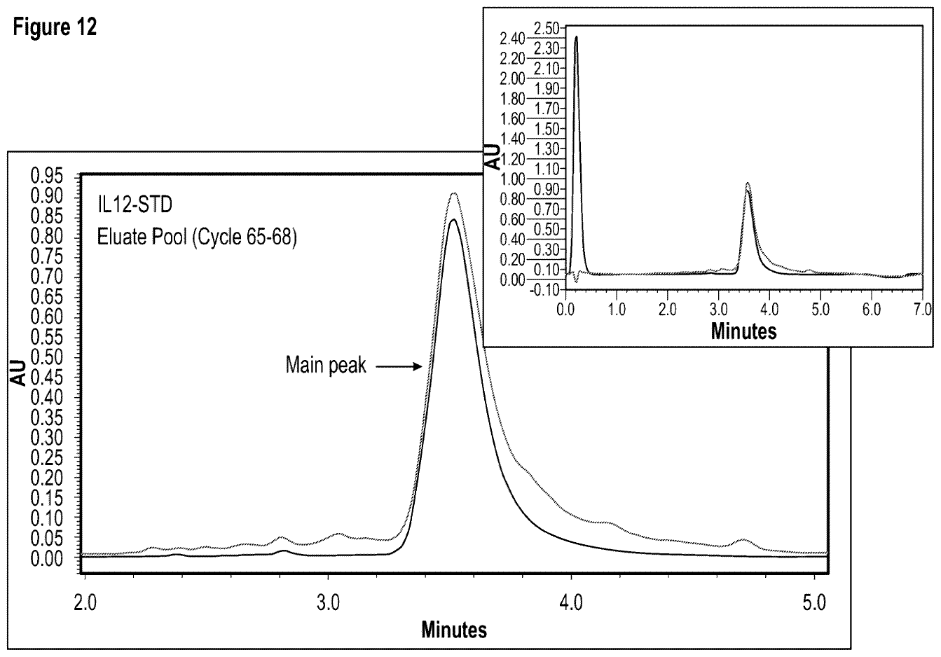

The operating parameters for the chromatographic process (such as flow rates and number of CVs passed, residence time) were not changed throughout the run except the number of cycles as the feed concentration is increased by 0.03 mg/L of harvest to 0.46 mg/mL of harvest during perfusion upstream process. The elution fraction samples were analyzed offline with RP-HPLC method for content and purity, respectively (Representative profile is shown in FIG. 12). A good amount of purification was observed after first capture step compared to reference standard as seen from FIG. 12. The yield was calculated by considering total protein in eluate fraction against loaded protein based on RP-UPLC data and average recovery of 66% was achieved (FIG. 11) The eluates from chromatography 1 were processed on phenyl Sepharose fast flow high sub resin (hydrophobic interaction chromatography) followed by POROS XS resin (cation exchange chromatography) for further control on the impurity profile. The final drug substance was prepared with UFDF followed sterile filtration after nano-filtration step was executed after cation exchange chromatography. The final drug substance was then compared with the reference standard material with RP-HPLC and SE-HPLC method. The operating process flow and parameters used for manufacturing scale batch executed for the IL-12 ABD fusion protein is shown in FIG. 9.

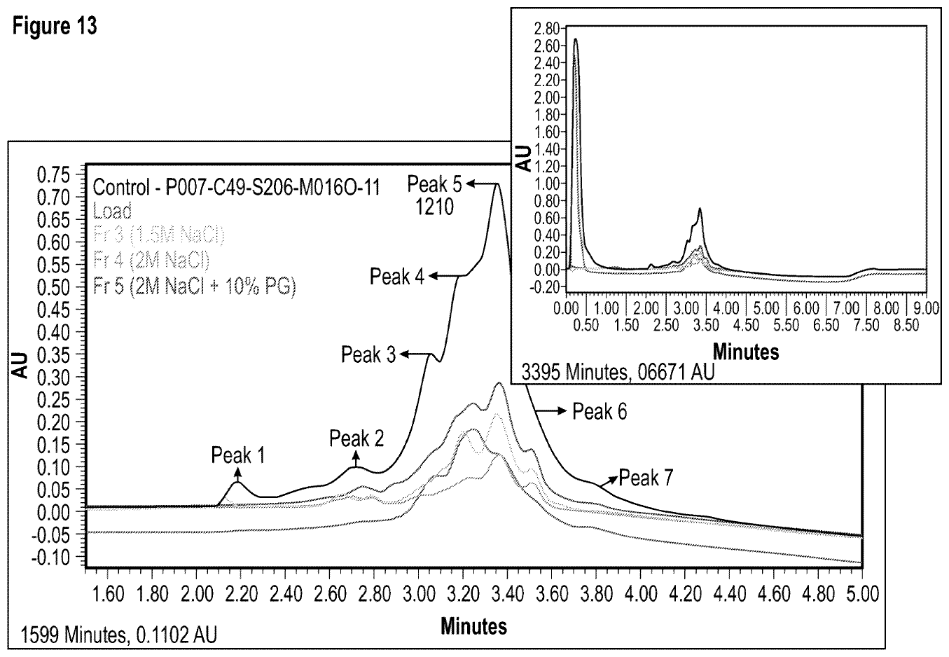

Capto™Blue affinity column was also tested for the purification of an IL-12/IL-15 ABD fusion protein (1210) that included the A10m3 albumin binding domain. Unlike the IL-12 ABD fusion protein, however, Capto™Blue did not selectively bind the IL-12/IL-15 ABD fusion protein in any of the elution conditions tested, and was present in all elution fractions (see FIG. 12 vs FIG. 13).

The examples set forth above are provided to give those of ordinary skill in the art a complete disclosure and description of how to make and use the embodiments of the compositions, systems and methods of the invention, and are not intended to limit the scope of what the inventors regard as their invention. Modifications of the above-described modes for carrying out the invention that are obvious to persons of skill in the art are intended to be within the scope of the following claims. All patents and publications mentioned in the specification are indicative of the levels of skill of those skilled in the art to which the invention pertains.

All headings and section designations are used for clarity and reference purposes only and are not to be considered limiting in any way. For example, those of skill in the art will appreciate the usefulness of combining various aspects from different headings and sections as appropriate according to the spirit and scope of the invention described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

1. A method of making a protein comprising:
a) culturing a plurality of mammalian host cells that each comprise a polynucleotide encoding for the protein in a bioreactor comprising a liquid culture medium,
wherein the host cells are cultured under conditions wherein the protein is produced and secreted by the host cells into the liquid culture medium,
b) removing liquid culture medium comprising the secreted protein from the bioreactor;
c) purifying the protein by passing the liquid culture medium removed from the bioreactor directly over one or more chromatography columns,
wherein the one or more chromatography columns are operably and continuously linked to the perfusion bioreactor; and
d) collecting the purified protein from the liquid culture medium,
wherein the protein is an IL-12 albumin binding domain (ABD) fusion protein.

2. The method of claim 1, wherein the bioreactor is a perfusion mode bioreactor.

3. The method of claim 2, wherein the liquid culture medium comprising the secreted protein is removed from the perfusion mode bioreactor and replaced with fresh liquid culture medium at set time intervals.

4. The method of claim 3, wherein the set time interval is 12 hours, 24 hours, 48 hours, or 72 hours.

5. The method of claim 2, wherein the liquid culture medium comprising the secreted protein is continuously removed from the perfusion mode bioreactor and continuously replaced with fresh liquid culture medium.

6. The method of claim 3, wherein the liquid culture medium is removed from the bioreactor starting at least about 96 hours after beginning the culturing step a).

7. The method of claim 3, wherein the liquid culture medium comprising the secreted protein is removed from the perfusion mode bioreactor and replaced with an equal volume of fresh liquid culture medium.

8. The method of claim 3, wherein a partial volume of the liquid culture medium comprising the secreted protein is partially removed from the perfusion mode bioreactor and replaced with an equal volume of fresh liquid culture medium.

9. The method of claim 1, wherein the host cells are prevented from leaving the perfusion bioreactor during the removing step b) using a cell separation system.

10. The method of claim 1, wherein the host cells are cultured in the presence of nutrients that are periodically replenished in the liquid culture medium.

11. The method of claim 1, wherein the plurality of mammalian host cells are cultured at a density of 20-100×10⁶ cells/mL of liquid culture medium in the bioreactor.

12. The method of claim 1, wherein the purifying c) separates one or more proteolytic enzymes from the protein.

13. The method of claim 1, wherein the albumin binding domain of the IL-12 ABD fusion protein comprises a variable heavy domain comprising SEQ ID NO:1 or a variant thereof, and a variable light domain comprising SEQ ID NO:5 or a variant thereof.

14. The method of claim 13, wherein the albumin binding domain is an scFv that comprises the amino acid sequence of SEQ ID NO:9 or a variant thereof.

15. The method of claim 1, wherein the IL-12 of the IL-12 fusion protein is a single chain IL-12 comprising a p35 subunit covalently attached to a p40 subunit.

16. The method of claim 15, wherein the p40 subunit has the amino acid sequence of SEQ ID NO:10 or a variant thereof.

17. The method of claim 15, wherein the p35 subunit has the amino acid sequence of SEQ ID NO: 11 or a variant thereof.

18. The method of claim 15, wherein the single chain IL-12 has the amino acid sequence of SEQ ID NO:12.

19. The method of claim 1, wherein the IL-12 ABD fusion protein has the amino acid sequence of SEQ ID NO:13.

20. The method of claim 1, wherein the one or more chromatography columns comprise an ion exchange chromatography column, a hydrophobic interaction column, an affinity column, a pseudo-affinity column or a size exclusion chromatography column.

21. The method of claim 20, wherein the one or more chromatography columns comprises an affinity column.

22. The method of claim 21, wherein the pseudo-affinity column comprises a Cibacron Blue ligand.

23. The method of claim 22, wherein the pseudo-affinity column comprises an affinity chromatography media depicted in FIG. 14.
